(19) United States
(12) Patent Application Publication
(10) Pub. No.: US 2007/0054964 A1
(43) Pub. Date: Mar. 8, 2007

(54) Topical formulations containing O-Desmethyl Venlafaxine (ODV) or its salts

(75) Inventors: Rao Tatapudy, Suffern, NY (US); Syed M. Shah, East Hanover, NJ (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/515,702

(22) Filed: Sep. 5, 2006

Related U.S. Application Data
(60) Provisional application No. 60/715,400, filed on Sep. 7, 2005.

(51) Int. Cl. A61K 31/138

(52) U.S. Cl. 514/651

(57) ABSTRACT

The present invention provides topical compositions comprising O-desmethylvenlafaxine (ODV), a selective serotonin and norepinephrine re-uptake inhibitor, or a pharmaceutically acceptable salt thereof. In certain embodiments, the inventive topical formulations contain one or more percutaneous/permucosal absorption enhancers. Also provided are methods of preparing and using these compositions for the treatment of diseases or conditions where a localized therapeutic effect is sought, such as vasomotor symptoms and pain.

### RELATED APPLICATIONS

The present application claims priority from Provisional Application No. 60/715,400 filed on Sep. 7, 2005 and entitled “Topical Formulations Containing O-Desmethyl Venlafaxine (ODV) or Its Salts”. The Provisional Application is incorporated herein by reference in its entirety.

### BACKGROUND OF THE INVENTION

Venlafaxine (or (±)-1-[2-(dimethylamino)-1-(4-methoxyphenyl)ethyl]-cyclohexanol) belongs to a relatively new class of anti-depressants (U.S. Pat. No. 4,761,501; J. T. Pento, Drugs of the future, 1988, 13: 839-840). Its hydrochloride salt is commercially available in the U.S. under the trade name Effexor® and is currently indicated for the treatment of depression and anxiety disorders.

In vivo, venlafaxine is extensively transformed by a saturable metabolic pathway into two minor metabolites, N-desmethylvenlafaxine and N,O-didesmethylvenlafaxine, and one major, biologically active metabolite, O-desmethylvenlafaxine (K. J. Klamerus et al., J. Clin. Pharmacol., 1992, 32: 716-724). Venlafaxine and O-desmethylvenlafaxine (ODV) are structurally unrelated to other anti-depressant drugs including tricyclic anti-depressants (TCAs), selective serotonin re-uptake inhibitors (SSRIs), monoamine oxidase inhibitors (MAOIs) and reversible inhibitors of monoamine oxidase (RIMAs). The mechanism of anti-depressant action of venlafaxine and ODV in humans is associated with their potentiation of neurotransmittor activity in the central nervous system. Venlafaxine and ODV have been shown to be potent inhibitors of neuronal serotonin and norepinephrine re-uptake and weak inhibitors of dopamine re-uptake. Selective serotonin and norepinephrine re-uptake inhibitors, or “SSNRIs”, i.e., compounds that exert their anti-depressant effect through the same mechanism as venlafaxine, have, in general, a quicker onset of therapeutic action and are usually more effective than other anti-depressants (J. S. Olver et al., CNS Drugs, 2001, 15: 941-954; M. E. Thase, J. Clin. Psychiatry, 64: 3-7; D. E. Stewart, J. Clin. Psychiatry, 2003, 64: 12-16). Furthermore, since Venlafaxine and ODV exhibit no significant affinity for muscarinic, H1-histaminergic or α1-adrenergic receptors, they are not associated with the various anticholinergic, sedative, and cardiovascular effects seen with other anti-depressant drugs.

Compared to venlafaxine, ODV possesses several advantageous properties. In addition to being more soluble than venlafaxine, ODV has been reported to have a half-life of about 10 hours, which is approximately 2.5 times as long as that of the parent compound (K. J. Klamerus et al., J. Clin. Pharmacol., 1992, 32: 716-724). In vitro studies suggest that ODV is also a more potent inhibitor of norepinephrine and serotonin re-uptake than venlafaxine (E. A. Muth et al., Drug Develop. Res., 1991, 23: 191-199). These advantages are all the more important given that ODV, like venlafaxine, can find applications in the treatment of other conditions than major depression.

For example, venlafaxine is known to be effective in treating obsessive-compulsive conditions, post-traumatic stress disorder, panic disorder, and other anxiety disorders (T. T. Pleak and L. J. Gormly, Am. J. Psychiatry, 1995, 152: 1099; T. D. Geracioti, J. Clin. Psychiatry, 1995, 56: 408-410; J. A. Yaryura-Tobias and F. A. Neziroglu, Arch. Gen. Psychiatry, 1996, 53: 653-654; D. Denys et al., J. Clin. Psychopharmacol., 2003, 23: 568-575; R. H. Bradley et al., Am. J. Ther., 2003, 10: 318-323; M. Katzman, Expert Rev. Neurother., 2004, 4: 371-381). Anti-depressants, such as venlafaxine, that block re-uptake of both serotonin and norepinephrine, have also been used to treat pain syndromes including, but not limited to, pain associated with major depression or an anxiety disorder (R. H. Bradley et al., Am. J. Ther., 2003, 10: 318-323); peripheral neuropathic pain (J. E. Sumpton and D. E. Moulin, Ann. Pharmacother., 2001, 35: 557-559; T. Tasmuth et al., Eur. J. Pain, 2002, 6: 17-24; S. Guldiken et al., Diabetes Nutr. Metab., 2004, 17: 247-249); chronic pain (K. Taylor and M. Rowbowtham, West. J. Med., 1996, 165: 147-148; D. A. Songer and H. Schulte, Am. J. Psychiatry, 1996, 153: 737; P. T. Ninan, Depress. Anxiety, 2000, 12: 90-94); cancer-related pain (J. P. Durand and F. Goldwasser, Anticancer Drugs, 2002, 13: 777-780; J. P. Durand et al., Anticancer Drugs, 2003, 14: 423-425; S. S. Reuben et al., J. Pain Symptom Manag., 2004, 27: 133-139), and fibromyalgia (M. M. Dwight et al., Psychosomatics, 1998, 39: 14-17; K. Sayar et al., Ann. Pharmacother., 2003, 37: 1561-1565). Venlafaxine is also considered as a promising non-hormonal alternative for relief of vasomotor symptoms (VMS) including hot flashes (C. L. Loprinzi et al., J. Clin. Oncol., 1998, 16: 2377-2381; S. K. Quella et al., J. Urol., 1999, 162L 98-102; D. H. Barlow, Lancet, 2000, 356: 2025-2026; C. L. Loprinzi et al., Lancet, 2000, 356: 2059-2063; D. Barton et al., Oncol. Nurs. Forum, 2002, 29: 33-40; A. N. Wymenga and D. T. Sleijfer, Acta Oncol., 2002, 41: 269-275; C. E. Schober and N. T. Ansani, Ann. Pharmacother., 2003, 37: 1703-1707), and ODV succinate is currently in Phase III clinical trials for VMS.

However, oral administration of venlafaxine is associated with adverse side effects including sustained hypertension, headache, asthenia, sweating, somnolence, dry mouth, dizziness, insomnia, nervousness, anxiety, blurred or blurry vision, sexual dysfunction (Physician's Desk Reference, 1999, 53^{rd} Ed, pp. 3293-3302; J. Sinclair et al., Rev. Contemp. Pharmacother., 1998, 9: 333-344), and, most commonly, gastrointestinal side effects such as nausea and vomiting (R. Entsuah and R. Chitra, Psychopharmacol. Bull., 1997, 33: 671-676). These adverse effects can significantly limit the dose level, frequency, and duration of treatment, and can even prevent the potential of such drugs from being fully realized.

There clearly exists a need for novel strategies for the administration of selective serotonin and norepinephrine re-uptake inhibitors such as ODV. Particularly desirable are delivery systems that would allow administration of therapeutically effective amounts of SSNRIs while avoiding or reducing the incidence, severity or duration of the undesired side effects generally associated with oral administration.

### SUMMARY OF THE INVENTION

The present invention is directed to systems and methods for the simple, convenient and non-invasive administration of ODV or its salts for the treatment of various diseases or conditions. More specifically, the present invention provides ODV topical compositions which offer the advantage of avoiding the gastrointestinal tract and hepatic first-pass biotransformation and metabolism. In particular, the inventive compositions allow for the rapid delivery of high concentrations of the drug, which results in fewer adverse side effects or drug-drug interactions than oral administration. The topical ODV compositions of the invention are particularly useful for the prevention, treatment or management of vasomotor symptoms and pain.

In one aspect, the present invention provides a topical composition comprising a therapeutically effective amount of ODV, or a pharmaceutically acceptable salt thereof, and at least one physiologically acceptable carrier or excipient. The topical composition may be formulated as an ointment, a cream, a lotion, a paste, a gel, a spray, an aerosol, or an oil. In certain embodiments, the topical composition is formulated as a cream or a gel.

In certain embodiments, the at least one physiologically acceptable carrier or excipient is selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

In some embodiments, the topical composition further comprises at least one absorption enhancer, such as pentadecalactone, 1,3-dioxalanes, 1,3-dioxanes, or any combination thereof.

In some embodiments, the topical composition further comprises a therapeutically effective amount of at least one pharmacologically active agent. The pharmacologically active agent may be selected from the group consisting of analgesics, anesthetics, muscle relaxants, neurotransmitter regulating agents, nociceptic agents, pre-menstrual medications, anti-menopausal agents, anti-aging agents, anti-anxiolytic agents, mood disorder agents, anti-depressants, anti-bipolar agents, anti-schizophrenic agents, tranquilizers, soporific agents, anti-migraine agents, skin temperature lowering products, anti-cancer agents, alkaloids, anti-metastatic agents, blood pressure controlling agents, hormones, steroids, anti-inflammatory agents, anti-ischemic agents, anti-arrhythmic agents, vitamins, minerals, anti-angiogenic agents, wound healing agents, cytokines, growth factors, anti-histaminic agents, anti-bacterial agents, anti-viral agents, antibiotics, counteracting appetite suppressants, dermatological agents such as skin renewal agents, sun screen and emollients, libido altering agents, laxatives, anti-diarrheic agents, antipruritic agents, antipyretic agents, immunostimulating agents, agents suitable for the treatment of prophylaxis diseases and conditions associated or accompanied with pain and/or inflammation, and any combination thereof.

The therapeutically effective amount of ODV, or a pharmaceutically acceptable salt thereof, present in a topical composition of the present invention is preferably between about 5 mg and about 500 mg, or between about 25 mg and about 250 mg, or between about 50 mg and about 200 mg, wherein the amount is calculated based on the amount of ODV free base. For example, in certain embodiments, the therapeutically effective amount of ODV, or a pharmaceutically acceptable salt thereof, is about 100 mg.

In another aspect, the present invention provides a method of treating vasomotor symptoms in a subject, the method comprising administering to the subject a therapeutically effective amount of a topical composition described herein.

In certain embodiments, the subject suffering from vasomotor symptoms experiences hot flashes, and administering the topical composition to the subject comprises applying a therapeutically effective amount of the composition to one or more skin surface areas of the subject's body experiencing hot flashes.

The method of the invention may be used to treat a female patient experiencing vasomotor symptoms associated with natural menopause, chemically-induced menopause or surgically-induced menopause. Alternatively or additionally, the inventive method may be used to treat a female patient who is receiving or has received breast cancer treatment, such as for example a treatment comprising administration of tamoxifen. The inventive method may also be used to treat a male patient who is naturally, chemically or surgically andropausal. Alternatively or additionally, the method may be used to treat a male patient who is being or has been treated for prostate cancer.

In still another aspect, the present invention provides a method of treating pain in a subject, the method comprising administering to the subject a therapeutically effective amount of an inventive topical composition. In certain embodiments, administering the topical composition to the subject comprises applying a therapeutically effective amount of the composition to one or more areas of the subject's body experiencing pain. The pain may be nociceptive pain or neuropathic pain.

These and other objects, advantages and features of the present invention will become apparent to those of ordinary skill in the art having read the following detailed description of the preferred embodiments.

### Definitions

Throughout the specification, several terms are employed that are defined in the following paragraphs.

The terms “individual”, “subject” and “patient” are used herein interchangeably. They refer to a higher vertebrate, preferably a human or another mammal (e.g., mice, rats, other rodents, rabbits, dogs, cats, cattle, swine, sheep, horses, or primates).

The terms “topical formulation” and “topical composition” are used herein interchangeably. They refer to a composition formulated such that the active ingredient(s) of the composition may be placed for direct application to a skin surface and from which an effective amount of the active ingredient(s) is released. Examples of topical formulations include, but are not limited to, ointments, creams, gels, lotions, sprays, pastes, and the like. In certain embodiments of the present invention, the compositions are formulated as creams or gels.

The terms “skin” and “skin surface” are used herein interchangeably. They encompass the skin surface of a subject comprising the epidermis as well as mucosal surfaces to which a composition of the present invention may be applied. Examples of mucosal surfaces include the mucosa of the respiratory, oral, vaginal, introital, labial, and rectal surfaces.

The term “transdermal” refers to the route of administration that facilitates transfer of the active ingredient(s) of a composition through a skin or mucosal surface and into the bloodstream.

The terms “penetration enhancer”, “permeation enhancer” and “absorption enhancer” are used herein interchangeably. They refer to compounds or substances that increase the permeability of skin or mucosa to a pharmacologically active agent so as to increase the rate at which the agent permeates through the skin or mucosa and enters the bloodstream. Absorption enhancers and their use in topical formulations are well known in the art.

A “pharmaceutical composition” is herein defined as comprising at least one physiologically acceptable carrier or excipient and an effective amount of ODV or a pharmaceutically acceptable salt thereof.

The term “ODV” refers to O-desmethylvenlafaxine (or 1-[2-(dimethyl-amino)-1-(4-phenyl)ethyl]-cyclohexanol), the major metabolite of venlafaxine.

As used herein, the term “pharmaceutically acceptable salt of ODV” refers to any salt of ODV derived from organic or inorganic acids, such as, for example, acetic, lactic, citric, cinnamic, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic acid, and the like, that is not toxic to the host at the concentrations at which it is administered. Preferably, a pharmaceutically acceptable salt of ODV has similar or superior biological activity than ODV and/or venlafaxine. Alternatively or additionally, a pharmaceutically acceptable salt of ODV exhibits desirable properties for topical administration (e.g., improved percutaneous/permucosal penetration). The term “pharmaceutically acceptable salt of ODV” also encompasses pharmaceutically acceptable salt hydrates of ODV (i.e., salts of ODV associated with molecules of water).

As used herein, the term “physiologically acceptable carrier or excipient” refers to a carrier medium or an excipient which does not interfere with the effectiveness of the biological activity of the active ingredient(s) of the composition and which is not excessively toxic to the host at the concentrations at which it is administered. In the context of the present invention, a physiologically acceptable carrier or excipient is preferably suitable for topical formulation. The term includes, but is not limited to, solvents, dispersion media, isotonic agents, percutaneous/permucosal absorption enhancers, and the like. The use of such media and agenets for the formulation of pharmaceutically active substances is well known in the art (see, for example, “Remington's Pharmaceutical Sciences”, E. W. Martin, 18^{th} Ed., 1990, Mack Publishing Co.: Easton, Pa., which is incorporated herein by reference in its entirety).

The term “treatment” is used herein to characterize a method that is aimed at (1) delaying or preventing the onset of a medical condition, disease or disorder; (2) slowing down or stopping the progression, aggravation, or deterioration of the symptoms of the condition; (3) bringing about ameliorations of the symptoms of the condition; and/or (4) curing the condition. The treatment may be administered prior to the onset of the condition, for a prophylactic or preventive action, or it may be administered after initiation of the condition, for a therapeutic action.

As used herein, the term “therapeutically effective amount” refers to an amount sufficient to achieve (in principle, for a subject of comparable characteristics, such as species, body type, size, extent of disease or disorder, degree or type of symptoms, history of responsiveness, and/or overall health) an intended biological or medical response or therapeutic benefit in a tissue, system or subject. For example, a desirable response may include one or more of: delaying or preventing the onset of a medical condition, disease or disorder, slowing down or stopping the progression, aggravation, or deterioration of the symptoms of the condition, bringing about ameliorations of the symptoms of the condition, and curing the condition. As will be appreciated by one skilled in the art, a therapeutically effective amount of ODV, or a pharmaceutically salt thereof, may be different depending on the desired response. For instance, an amount of ODV effective to treat pain may be different from an amount of ODV effective to treat vasomotor symptoms. Similarly, an amount of ODV effective to prevent vasomotor symptoms may be different from an amount of ODV effective to treat vasomotor symptoms, and either may be different from amounts to prevent or treat pain. It will also be appreciated that an amount of ODV effective to treat a local condition (e.g., pain) may be different from an amount of ODV effective to treat a condition where systemic drug distribution is desired (e.g., vasomotor symptoms).

Furthermore, when a combination of the present invention comprises ODV and other therapeutic agents, the amount of any individual agent required in the combination may be different from the amount required of that agent to achieve its therapeutic effect alone. In some cases, synergies between or among therapeutic agents used in a combination may reduce amounts required; in other cases, inhibitory interactions may increase amounts required. Thus, in general, therapeutically effective amounts of a combination of agents may utilize different absolute amounts of the agents than what constitute therapeutically effective amounts of the agents individually.

As used herein, the term “co-administration” refers to administration of multiple biologically active substances to one subject, either simultaneously or sequentially. The term also refers to the simultaneous or sequential administration of a single biologically active substance to one subject using different administration routes (e.g., orally and topically).

The term “about” is used herein to mean within 10%, preferably within 5%, and more preferably within 1% of a given value or range. Alternatively, the term “about” means within an acceptable standard error of the mean, when considered by one of ordinary skill in the art.

The term “hotflash” has herein its art understood meaning and refers to an episodic disturbance in body temperature typically consisting of a sudden skin flushing, usually accompanied with perspiration.

The terms “vasomotor symptoms”, “vasomotor instability symptoms” and “vasomotor disturbances” are used herein interchangeably and include, but are not limited to, hot flashes, insomnia, sleep disturbances, mood disorders, irritability, excessive perspiration, night sweats, fatigue, and the like, caused by thermoregulatory dysfunction.

As used herein, the term “pain” refers to any type of nociceptive pain or neuropathic pain, whether centralized or localized.

### DETAILED DESCRIPTION OF CERTAIN PREFERRED EMBODIMENTS

As mentioned above, the present invention provides topical compositions comprising ODV, or a pharmaceutically acceptable salt thereof, which can be useful for the prevention, treatment or management of vasomotor symptoms and/or pain.

I—ODV and Pharmaceutically Acceptable Salts

In certain embodiments, the topical compositions of the present invention comprise ODV as active ingredient. ODV free base is a colorless solid; its preparation and physicochemical characteristics have been described in International Patent Applications WO00/32555 and WO 00/59851 (each of which is incorporated herein by reference in its entirety).

ODV contains an asymmetric carbon atom. Accordingly, in the topical compositions of the present invention, ODV may be present as the racemic mixture, as a non-equimolar mixture of the (+) and (−) enantiomeric forms of ODV, as the stereoisomerically pure (+) enantiomer or as the stereoisomerically pure (−) enantiomer. The term “stereoisomerically pure”, as used herein, refers to compounds which are comprised of a greater proportion of the desired isomer than the racemic mixture. A stereoisomerically pure compound is preferably made up of at least about 90% of the desired isomer, more preferably of at least 95% of the desired isomer, even more preferably of more than 97% of the desired isomer.

In certain topical compositions of the present invention, the active ingredient is a pharmaceutically acceptable salt of ODV. Preferred salts for use in the preparation of topical compositions according to the present invention are pharmaceutically acceptable acid addition salts of ODV. These salts may be prepared by conventional methods which are well known in the art, for example, by reacting ODV free base with an equivalent amount of any acid that leads to the formation of a non-toxic salt. Suitable acids include organic and inorganic acids, such as, for example, acetic, lactic, citric, cinnamic, succinic, fumaric, maleric, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic acid, and the like.

ODV salts used in the preparation of topical compositions of the present invention may be crystalline or under a polymorphic or amorphous form. Hydrates as well as anhydrous forms of the salts are also encompassed by the present invention.

Several salts of ODV have been prepared, including the fumarate (U.S. Pat. No. 4,535,186) and succinates (U.S. Pat. No. 6,673,838), that have different physicochemical (e.g., solubility, stability and hygroscopy) and biological characteristics than ODV free base. For example, ODV succinate has been shown to exhibit improved solubility, permeability and bioavailability, and its oral administration has been found to result in a lower incidence of nausea, vomiting, diarrhea, abdominal pain, headache, vaso-vagal malaise, and/or trismus than oral administration of venlafaxine, ODV or other salts of ODV.

Selecting a pharmaceutically acceptable salt of ODV for the preparation of a topical composition of the present invention may readily be performed by one of ordinary skill in the art.

II—Formulation of ODV Topical Compositions

ODV topical compositions according to the present invention may be in the form of liquid or semi-solid dosage preparations. For example, inventive ODV compositions may be formulated as solutions, dispersions, suspensions, emulsions, mixtures, lotions, liniments, jellies, ointments, creams, pastes, gels, hydrogels, aerosols, sprays, plasters, bandages, sheets, foams, films, sponges, dressings, drenches, bioadsorbable patches, and sticks. In certain preferred embodiments of the present invention, ODV compositions are formulated as creams or gels.

The inventive topical compositions may be prepared according to general pharmaceutical practice (see, for example, “Remington's Pharmaceutical Sciences”, E. W. Martin, 18^{th} Ed., 1990, Mack Publishing Co.: Easton, Pa. and “Encyclopedia of Pharmaceutical Technology”, 1988, J. Swarbrick, and J. C. Boylan (Eds.), Marcel Dekker, Inc: New York, each of which is incorporated herein by reference in its entirety).

ODV topical compositions of the present invention preferably comprise a therapeutically effective amount of ODV, or a pharmaceutically acceptable salt thereof, and at least one physiologically acceptable carrier, vehicle or excipient. Physiologically acceptable carriers, vehicles, and/or excipients suitable for incorporation into topical compositions of the present invention can be routinely selected for a particular use by those skilled in the art. Such carriers, vehicles, and excipients include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, emollients, and skin protecting agents.

Examples of solvents are water or purified water, alcohols (e.g., ethanol, benzyl alcohol), vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, diethylamine, sodium hydroxide and tromethane (i.e., tris(hydroxymethyl)aminomethane hydrochloride). Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a topical composition of the invention to prevent microbial contamination that can affect the stability of the formulation and/or cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin, sorbitan mono-oleate derivatives), sorbitan esters, mono glycerides, fatty alcohols (e.g., cetyl alcohol, oleyl alcohol), and fatty acid esters (e.g., triglycerides of fatty acids, sodium cetostearyl sulfate). Anti-foaming agents usually facilitate manufacture of pharmaceutical compositions, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, propylene carbonate, carboxyvinyl polymers, magnesium-aluminum silicates, hydrophilic polymers (such as, for example, starch or cellulose derivatives), water-swellable hydrocolloids, carragenans, hyaluronates, alginates, and acrylates. Ointment bases suitable for use in the compositions of the present invention may be hydrophobic or hydrophilic, and include paraffin, lanolin, liquid polyalkylsiloxanes, cetanol, cetyl palmitate, vegetal oils, sorbitan esters of fatty acids, polyethylene glycols, and condensation products between sorbitan esters of fatty acids, ethylene oxide (e.g., polyoxyethylene sorbitan monooleate), polysorbates, white petrolatum and white wax.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

Alternatively or additionally, ODV topical compositions of the present invention may further comprise other types of excipients including thickening agents, bioadhesive polymers, and permeation enhancing agents.

Thickening agents are generally used to increase viscosity and improve bioadhesive properties of pharmaceutical or cosmetic compositions. Examples of thickening agents include, but are not limited to, celluloses, polyethylene glycol, polyethylene oxide, naturally occurring gums, gelatin, karaya, pectin, alginic acid, povidone, and Carbopol® polymers. Particularly interesting are thickening agents with thixotropic properties (i.e., agents whose viscosity is decreased by shaking or stirring). The presence of such an agent in a composition allows the viscosity of the composition to be reduced at the time of administration to facilitate its application to the skin and, to increase after application so that the composition remains at the site of administration.

Bioadhesive polymers are useful to hydrate the skin and enhance its permeability. Bioadhesive polymers can also function as thickening agents. Examples of bioadhesive polymers include, but are not limited to, pectin, alginic acid, chitosan, polysorbates, poly(ethyleneglycol), oligosaccharides and polysaccharides, cellulose esters and cellulose ethers, and modified cellulose polymers.

Permeation enhancing agents are vehicles containing specific agents that affect the delivery of active components through the skin. Permeation enhancing agents are generally divided into two classes: solvents and surface active compounds (amphiphilic molecules). Examples of solvent permeation enhancing agents include alcohols (e.g., ethyl alcohol, isopropyl alcohol), dimethyl formamide, dimethyl acetamide, dimethyl sulfoxide, 1-dodecylazocyloheptan-2-one, N-decyl-methylsulfoxide, lactic acid, N,N-diethyl-m-toluamide, N-methylpyrrolidone, nonane, oleic acid, petrolatum, polyethylene glycol, propylene glycol, salicylic acid, urea, terpenes, and trichloroethanol. Surfactant permeation enhancing agents may be nonionic, amphoteric, cationic, or zwitterionic. Suitable nonioinic surfactants include poly(oxyethylene)-poly(oxypropylene) block copolymers, commercially known as poloxamers; ethoxylated hydrogenated castor oils; polysorbates, such as Tween 20 or Tween 80. Amphoteric surfactants include quaternized imidazole derivatives, cationic surfactants include cetypyridinium chloride, and zwitterionic surfactants include the betaines and sulfobetaines. Other examples of suitable permeation enhancers include pentadecalactone, 2-pyrrolidine, 1-dodecal-azacycloheptane-2-one, calcium thioglycolate, hexanol, derivatives of 1,3-dioxanes (i.e., 1,3-dioxacyclohexanes) and 1,3-dioxalanes (i.e., 1,3-dioxacyclopentanes), 1-N-dodecyl-2-pyrrolidone-5-carboxylic acid, 2-pentyl-2-oxo-pyrrolidineacetic acid, 2-dodecyl-2-oxo-1-pyrrolidineacetic acid, and 1-azacycloheptan-2-one-2-dodecylacetic acid among others.

In certain embodiments of the present invention, ODV topical compositions are formulated to provide a local controlled release of one or more components of the composition. Any pharmaceutically acceptable carrier vehicle or formulation suitable for local administration may be employed. Examples of slow release formulation include coated-pellets, polymer formulations (such as vesicles or liposomes), microparticles (e.g., microspheres or microcapsules).

A wide variety of biodegradable materials may be used to provide controlled release of one or more components of the inventive compositions. The controlled release material should be biocompatible and be degraded, dissolved or absorbed in situ in a safe and pharmaceutically acceptable manner so that the material is removed from the site of administration by natural tissue processes and in a suitable amount of time (e.g., less than one year, preferably less than six months, and most preferably less than one month). The controlled release carrier should not cause any unwanted local tissue reaction or induce systemic or local toxicity.

Suitable controlled release biodegradable polymers for use in the formulation of topical compositions of the invention may comprise polylactides, polyglycolides, poly(lactide-co-glycolides), polyanhydrides, polyorthoesters, polycaprolactones, polysaccharides, polyphosphazenes, proteinaceous polymers and their soluble derivatives (such as gelation biodegradable synthetic polypeptides, alkylated collagen, and alkylated elastin), soluble derivatives of polysaccharides, polypeptides, polyesters, and polyorthoesters.

The pharmacokinetic release profile of these formulations may be first order, zero order, bi- or multi-phasic, to provide the desired therapeutic effect (e.g., pain relief) over the desired period of time. A desired release profile can be achieved by using a mixture of polymers having different release rates and/or different percent loadings of ODV, or a pharmaceutically acceptable salt thereof. Methods for the manufacture of coated-pellets, liposomes, microspheres and microcapsules are well known in the art.

III—Additional Biologically Active or Therapeutic Agents

As already mentioned above, ODV topical compositions of the present invention can be used to treat vasomotor symptoms and/or pain. In certain embodiments, ODV is combined with one or more additional pharmaceutically active agents. More specifically, topical compositions are provided herein that comprise ODV, or a pharmaceutically acceptable salt thereof, at least one physiologically acceptable carrier or excipient, and a therapeutically effective amount of at least one pharmacologically active agent. When applied to the skin or mucosa surface to be treated, the topical composition acts as a delivery system for the additional agent(s) it contains.

In certain embodiments of the invention, the additional pharmacologically active agent has pain-relief activity. Alternatively or additionally, the pharmacologically active agent may relieve one or more side effects associated with a pain-relieving agent contained in the composition, or may relieve one or more other symptoms or conditions associated with the pain or otherwise of concern to the subject suffering from or susceptible to pain. In other embodiments of the invention, the additional pharmacologically active agent is selected for its ability to directly or indirectly prevent, alleviate or reduce vasomotor symptoms.

An ODV topical composition of the present invention may comprise a single additional pharmacologically active agent, or, alternatively, it may comprise more than one additional active agent. A pharmacologically active agent may exhibit a single desirable property or more than one desirable property. As will be appreciated by one of ordinary skill in the art, a large variety of ODV topical compositions may be produced according to the present invention. The design of such compositions will mainly depend on their intended purpose(s), as well as on the desired additional or enhanced therapeutic effect(s) of the composition (e.g., anti-inflammatory or anesthetic activity).

Pharmacologically active agents suitable for incorporation into ODV topical compositions of the present invention include, but are not limited to, analgesics, anesthetics, muscle relaxants, neurotransmitter regulating agents, nociceptic agents, pre-menstrual medications, anti-menopausal agents, anti-aging agents, anti-anxiolytic agents, mood disorder agents, anti-depressants, anti-bipolar agents, anti-schizophrenic agents, tranquilizers, soporific agents, anti-migraine agents, skin temperature lowering products, anti-cancer agents, alkaloids, anti-metastatic agents, blood pressure controlling agents, hormones, steroids, anti-inflammatory agents, anti-ischemic agents, anti-arrhythmic agents, vitamins, minerals, anti-angiogenic agents, wound healing agents, cytokines, growth factors, anti-histaminic agents, anti-bacterial agents, anti-viral agents, antibiotics, appetite suppressants, dermatological agents such as skin renewal agents, sun screen and emollients, libido altering agents, laxatives, anti-diarrheic agents, antipruritic agents, antipyretic agents, immunostimulating agents, and other agents suitable for the treatment of prophylaxis diseases and conditions associated or accompanied with pain and/or inflammation. Specific examples of suitable pharmacologically active agents are provided and discussed below.

Pain Relieving Agents

In those embodiments where an inventive ODV topical composition is to be used for the prevention, treatment or management of pain, the composition may further comprise a therapeutically effective amount of at least one pain-relieving agent.

There are two types of pain: nociceptive pain and neuropathic pain. Nociceptive pain has been defined as an appropriate physiological response to a painful stimulus. It is caused by noxious stimulation of peripheral nerve endings (i.e., nociceptors), which then transmit impulses over intact neural pathways to the spinal neurons and then to the brain. Nociceptive pain may occur as a result of inflammation, injury, disease or muscle spasm. Neuropathic pain has been defined as an inappropriate response caused by a primary lesion or dysfunction in the nervous system. It is generally caused by damage to neural structures, mainly to nociceptors, which become extremely sensitive and can generate impulses in the absence of stimulation. Nociceptor damage may be due to, for example, trauma, infection, metabolic disorder or cancer. Neuropathic pain is a major factor in the development of chronic pain, and may be associated with pathological states where there is a reduction in pain threshold (i.e., allodynia), an increased response to noxious stimuli (hyperalgesia), or an increased response duration (persistent pain).

The present invention provides ODV topical compositions, as described herein, that further comprise a therapeutically effective amount of at least one pain-relieving agent. Pain-relievers suitable for incorporation into ODV topical compositions include, but are not limited to, substances, molecules, agents or drugs which, when applied topically, have a temporary analgesic, anesthetic, numbing, paralyzing, relaxing, and/or calming effect.

Analgesics suitable for use in the present invention include non-steroidal, anti-inflammatory drugs (NSAIDs). NSAIDs have analgesic, antipyretic and anti-inflammatory activity. They act peripherally to provide their analgesic effect by interfering with the synthesis of prostaglandin, through cyclooxygenase (COX) inhibition. There are many different types of NSAIDs, including aspirin and other salicylates. Examples include, but are not limited to, ibuprofen, naproxen, sulindac, diclofenac, piroxicam, ketoprofen, diflunisal, nabumetone, etodolac, oxaprozin, and indomethacin. Aspirin acts as an anti-inflammatory agent when administered in high doses, otherwise it is just a pain killer like acetaminophen. Acetaminophen has similar analgesic and antipyretic effects to the NSAIDs, but does not provide an anti-inflammatory effect. Several of the more potent NSAIDs have been developed into topical products for local applications to painful areas of the body.

Analgesics suitable for use in the present invention also include opioids. As used herein, the term “opioid” refers to any agonists or antagonists of opioid receptors such as the μ-, κ-, and δ-opioid receptors and different subtypes. Some opioids exhibit a high affinity for one of the opioid receptors, while others interact with more than one receptors.

Opioid analgesics are classified as full agonists, partial agonists, or mixed agonists-antagonists, depending on the specific receptors to which they bind and their intrinsic activity at that receptor. Commonly used full agonists include morphine, hydromorphone, codeine, oxycodone, hydrocodone, methadone, levarphanol, and fentanyl. These opioids are classified as full agonists because they do not have a ceiling effect to their analgesic efficacy and do not reverse or antagonize the effects of other opioids within this class if given simultaneously. Bupernorphine is a partial agonist; it has a relatively low intrinsic efficacy at the opioid receptors in comparison to full opioid agonists and displays a ceiling effect to analgesia. Mixed agonist-antagonists in clinical use include pentazocine, butrophanol tartrate, dezoxine, and nalbuphine hydrochloride. In contrast to full agonists, these drugs have an analgesic ceiling and block opioid analgesia at one type of opioid receptor (μ) or are neutral at this receptor while simultaneously activating a different opioid receptor (κ).

Opioids that can be used in the practice of the present invention include all agonists and antagonists with morphine-like activity; naturally occurring endogenous and synthetic opioid peptides; and opiates (i.e., drugs which are derived from opium, such as morphine, codeine and a wide variety of semi-synthetic opioid congeners derived from these compounds and from thebaine, another component of opium).

Examples of suitable opioids include, but are not limited to, alfentanil, allylprodine, alphaprodine, amiphenazole, anileridine, benzeneacetamine, benzoylhydrazone, benzylmorphine, benzitramide, nor-binaltorphimine, bremazocine, buprenorphine, butorphanol, clonitazene, codeine, cyclazocine, desomorphine, dextromoramide, dezocine, diampromide, dihydrocodeine, dihydrocodeine enol acetate, dihydromorphine, dimenoxadol, dimepheptanol, dimethyl-thiambutene, dioxaphetyl butyrate, dipipanone, diprenorphine, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levallorphan, levorphanol, lofentanil, loperamide, meperidine, meptazinol, metazocaine, methadone, metopon, morphine, morphiceptin, myrophine, nalbuphine, nalmefene, nalorphine, naltrindole, naloxone, naltrexone, narceine, nicomorphine, norlevorphanol, normethadone, normorphine, norpipanone, opium, oxycodone, oxymorphone, papaveretum, papaverine, pentazocine, phenadoxone, phenazocine, phenoperidine, piminodine, piperidine, pirtramide, proheptazine, promedol, propiram, propoxyphene, remifentanil, spiradoline, sufentanil, tilidine, trifluadom, and active derivatives, prodrugs, analogs, pharmaceutically acceptable salts, or mixtures thereof.

Examples of suitable peptide opioids include, but are not limited to, [Leu⁵]enkephalin, [Met⁵]enkephalin, DynorphinA, Dynorphin B, α-Neoendorphin, β-Neoendorphin, βₕ-Endorphin, Deltorphin II, Morphiceptin, and active derivatives, analogs, pharmaceutically acceptable salts, or mixtures thereof.

Since synergy is known to occur between opioids of different classes (J. U. Adams et al., J. Pharmacol. Exp. Ther., 1993, 266: 1261-1267; L. He and N. M. Lee, J. Pharmacol. Exp. Ther., 1998, 285: 1181-1186; G. C. Rossi et al., Brain Res., 1994, 665: 85-93), in certain embodiments, the topical compositions of the present invention comprise ODV, or a salt thereof, as described above and a therapeutically effective amount of two or more opioid analgesics.

Opioids are also known to work in combination with other classes of drugs (see, for example, U.S. Pat. Nos. 5,840,731 and 5,869,498; and WO 97/10815). Adjuvant drugs may be used to enhance the analgesic efficacy of opioids, treat concurrent symptoms that exacerbate the pain, or provide independent analgesia for specific types of pain. Agents that may be used as adjuvant drugs include, but are not limited to, local anesthetics, antidepressants, anticonvulsants, and corticosteroids.

Anesthetics, such as xylocalne, lidocaine or benzocaine (or other drugs such as those described below) may be added to inventive ODV topical compositions to provide an immediate but short-term pain relief until ODV and/or another analgesic agent present in the compositions become(s) fully effective.

Anesthetics that are suitable for use in the practice of the present invention include sodium-channel blockers. Sodium-channel blockers prevent the generation and conduction of nerve impulses by decreasing or preventing the large transient increase in the permeability of excitable membranes to sodium ions, Na⁺. Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, etidocaine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolamine, and active derivatives, prodrugs, analogs, pharmaceutically acceptable salts, or mixtures thereof.

In order to improve the effectiveness and tolerance of an inventive ODV topical composition, local anesthetics with different pharmacodynamics and pharmacokinetics may be combined in the composition. Accordingly, in certain embodiments, the composition comprises ODV, or a salt thereof, as described above, and a therapeutically effective amount of two or more anesthetic agents. For example, a preferred combination of anesthetic agents is an eutectic mixture of lidocaine and prilocalne. Another preferred combination is a mixture of lidocaine and tetracaine.

In other embodiments of the present invention, the ODV topical composition further comprises a therapeutically effective amount of an agent that can prolong the local anesthetic effect and/or enhance the effectiveness of the local anesthetic agent(s) contained in the composition.

It has been reported (see, for example, U.S. Pat. Nos. 5,922,340 and 6,046,187) that the co-administration of a glucocorticosteroid may prolong or otherwise enhance the effect of local anesthetics. Glucocorticosteroids that may be used in the inventive compositions include dexamethazone, cortisone, hydrocortisone, prednisone, prednisolone, beclomethasone, betamethasone, flunisolide, fluocinolone, acetonide, fluocinonide, triamcinolone, and the like.

Locally acting vasoconstrictive agents are also known to provide effective enhancement of local anesthesia, especially when administered through controlled release. Vasoconstrictor agents include, but are not limited to, catechol amines (e.g., epinephrine, norepinephrine and dopamine); metaraminol, phenylephrine, sumatriptan and analogs, α-1 and α-2 adrenergic agonists, such as, for example, clonidine, guanfacine, guanabenz, and dopa (i.e., dihydroxyphenylalanine), methyldopa, ephedrine, amphetamine, methamphetamine, methylphenidate, ethylnorepinephrine ritalin, pemoline, and other sympathomimetic agents.

Other adjuvant drugs that can be used in the present invention include N-methyl-D-aspartate (“NMDA”) receptor antagonists (such as ketamine), which are known to have local anesthetic properties. In addition to ketamine, NMDA-receptor antagonists include dextro-methorphan, dextrorphan, pyroloquinoline quinone, cis-4-(phosphonomethyl)-2-piperidine carboxylic acid, MK801, and memantine.

Anti-Inflammatory Agents

Inflammation is a natural consequence of injury of adult tissues and the body's initial attempt at healing itself. While the inflammatory response is essential to healing, severe, prolonged inflammation can perpetuate pain. The present invention provides ODV topical compositions, as described herein, that further comprise a therapeutically effective amount of at least one anti-inflammatory agent. Anti-inflammatory agents suitable for use in the present invention are substances, molecules or drugs, which, when applied topically, have an anti-inflammatory activity (i.e., they can prevent or reduce the duration and/or severity of inflammation; prevent or reduce injury to cells or damage to tissue caused by inflammation; and/or provide relief from at least one of the manifestation of inflammation such as erythema, swelling, tissue ischemia, itching, fever, and the like).

Anti-inflammatory agents suitable for use in the present invention may be selected from a wide variety of steroidal and non-steroidal anti-inflammatory agents.

Examples of NSAIDs can be found above. Examples of steroidal anti-inflammatory agents include, but are not limited to, aclomethasone dipropionate, flunisolide, fluticasone, budesonide, triamcinolone, triamcinoline acetonide, beclomethasone diproprionate, betamethasone valerate, betamethasone diproprionate, hydrocortisone, cortisone, dexamethason, mometasone furoate, prednisone, methylprednisolone aceponate, and prednisolone. Steroids are synthetic forms of naturally occurring hormones produced by the adrenal glands. They can provide rapid and powerful reduction of pain and inflammation by stopping the production of prostaglandins. Topical administration of steroids avoids the side effects which are generally associated with their systemic administration including blood sugar elevations, hypertension, osteoporosis, and weight gain.

Alternatively or additionally, anti-inflammatory agents may be selected from the wide variety of substances, molecules, and drugs exhibiting antioxidant activity. Antioxidants are agents that can prevent or reduce oxidative damage caused to tissue by inflammatory processes that involve the production of reactive oxygen species (ROS). Antioxidants suitable for incorporation into ODV topical compositions of the present invention are substances, molecules, or drugs that can prevent, inhibit or suppress biological damage associated with reactive oxygen species. These include agents that can scavenge ROS; agents that can limit the production of ROS by activated neutrophils or macrophages, for example, by inhibiting the respiratory burst; agents that can reduce the number of neutrophils or macrophages attracted to the site of inflammation; and agents that effect their antioxidant activity by any combinations of these mechanisms of action.

Examples of suitable antioxidants include, but are not limited to, vitamin A (retinal), vitamin B (3,4-didehydroretinol), vitamin C (D-ascorbic acid, L-ascorbic acid), α-carotene, β-carotene, γ-carotene, δ-carotene, vitamin E (α-tocopherol), β-tocopherol, γ-tocopherol, δ-tocopherol, tocoquinone, tocotrienol, butylated hydroxy anisole, cysteine, and active derivatives, analogs, precursors, prodrugs, pharmaceutically acceptable salts or mixtures thereof.

An anti-inflammatory ODV topical composition of the invention may further comprise a topical antipruritic agent such as menthol, and/or a decongestant such as eucalyptus oil.

Anti-Cancer Agents

As already mentioned above, cancer is often associated with pain. Accordingly, the present invention provides ODV topical compositions further comprising a therapeutically effective amount of at least one chemotherapeutic anti-cancer agent. These inventive compositions may, for example, be applied to a surgical site from which a tumor has been ablated to alleviate pain and prevent regrowth from any residual tumor cells, after closure of the surgical wound.

The regional delivery of anti-cancer agents is not a new concept. Following the recognition in the 1950s that cytotoxic alkylating drugs could cause shrinkage of tumors in individuals with advanced ovarian cancer, these agents have been directly instilled into the peritoneal cavity in an effort to treat the malignancy (A. S. Weisberger et al., JAMA, 1955, 159: 1701-1707). Since then, different therapeutic strategies including the intrathecal administration or methotrexate in the treatment and prevention of meningeal leukemia (W. A. Bleyer, Natl. Cancer Inst. Monogr., 1977, 46: 171-178), intravesical treatment of superficial bladder cancer (H. C. Jones et al., Lancet, 1961, 2: 615-618), and direct administration of drugs into the blood vessels feeding a localized cancer tumor (D. B. Calvo et al., Cancer, 1980, 45: 1278-1283) have been evaluated. One advantage of local delivery of antineoplastic agents is the possibility to increase the concentrations of drugs at the tumor site while limiting systemic toxicity.

Chemotherapeutic anti-cancer agents suitable for incorporation in ODV topical compositions of the present invention are substances, molecules, or drugs which, when applied locally, can prevent or reduce cancer cell proliferation, destroy cancer cells, and/or prevent or reduce metastasis.

Examples of chemotherapeutic anti-cancer agents include, but are not limited to, alitretinoin, altretamine, bexarotene, capecitabine, carmustine with Polifeprosan 20 Implant (Gliadel Wafer), cisplatin, cytarabine liposomal (DepoCyt), cyclophosphamide, daunorubicin liposomal, docetaxel, doxorubicin liposomal, epirubin, etoposide phosphate, 5-fluorouracil, gemcitabine, gemtuzumab-ozogamicin, imatinib mesylate (Gleevec), irinotecan, oxaliplatin, levamisole, navelbine, mitoguazone, mitomycin, mitoxantrone, paclitaxel, temozolamide, topotecan, triapine, trimetrexate, somatuline, valrubicin, and vinblastine.

Other Pharmacologically Active Agents

As already mentioned above, the inventive ODV topical compositions may be used for the prevention, treatment or management of vasomotor symptoms.

Vasomotor symptoms (VMS), which include hot flashes and night sweats, are the most common symptoms associated with menopause, occurring in 60% to 80% of all women following natural, or chemically- or surgically-induced menopause (H. L. Judd et al., Obstet. Gynecol., 1981, 58: 267-275). A hot flash is characterized by a heat-dissipation response that consists of the sudden onset of sweating of the face, neck and chest, as well as peripheral withdrawal vasodilation (R. R. Freedman, Am. J. Human Biol., 2001, 13: 453-464). Hot flashes can last up to 30 minutes and vary in their frequency from several times a week to multiple occurrences per day. Often dizziness, palpitations and diaphoresis accompany such episodes, which can lead to sleep disruption and interfere with the quality of life. Vasomotor symptoms are often even more severe in women treated for breast cancer, in particular in those patients who are given the anti-estrogen drug tamoxifen. Men also experience hot flashes following steroid hormone (androgen) withdrawal, in cases of age-associated androgen decline as well as in extreme cases of hormone deprivation associated with treatment for prostate cancer (H. H. Berendsen et al., Eur. J. Pharmacol., 2001, 419: 47-54). As many as one-third of these prostate cancer patients experience persistent and frequent symptoms severe enough to cause significant discomfort and inconvenience.

In those embodiments where an ODV topical compositions of the present invention is to be used for the prevention, treatment or management of vasomotor symptoms or vasomotor instability, the composition may further comprise a therapeutically effective amount of at least one pharmacologically active agent selected for its ability to prevent, reduce or alleviate one or more vasomotor symptoms. Alternatively or additionally, a pharmacologically active agent may be selected for its ability to relieve one or more other symptoms or conditions associated with VMS or otherwise of concern to the subject suffering from VMS.

The most commonly used treatments for hot flashes are hormone-replacement therapy (HRT; estrogen and progesterone) and estrogen-replacement therapy (ERT). Accordingly, in certain embodiments, the ODV topical compositions of the present invention further comprise a therapeutically effective amount of at least one hormone known to be useful in the management of vasomotor symptoms. Suitable hormones include estrogens, progestins, and androgens.

The term “estrogen”, as used herein, refers to any substance, natural or synthetic, that exerts a biological or pharmacological action primarily by binding to estrogen receptors. Examples of suitable estrogens include, but are not limited to, 17-β-estradiol, 17-α-estradiol, estriol, estrone, and phytoestrogens. These substances may be derived or modified to form, for example, conjugated estrogens, esterified estrogens, ethinyl estradiol, etc. Also suitable are selective estrogen receptor modulators such as raloxifene and the like. Estrogenic hormones incorporated into an inventive ODV topical composition may be present as salts (e.g., sodium estrogen sulfate), isomers, or prodrugs. Examples of phytoestrogens (i.e., plant-derived estrogens) include isoflavones such as genistein, diaszein and equol.

The term “progestin”, as used herein, refers to any substance, natural or synthetic, that exerts a biological or pharmacological action primarily by binding to progestin receptors. Examples of suitable progestins for use in ODV topical compositions of the present invention include, but are not limited to, progesterone, medroxy-progesterone acetate, norethindrone, and norethindrone acetate, esters, derivatives, prodrugs, and isomers thereof.

The term “androgen”, as used herein, refers to a steroid, natural or synthetic, which exerts its biological or pharmacological action primarily by binding to androgen receptors. Examples of suitable androgens for incorporation into the inventive compositions include, but are not limited to, testosterone, methyltestosterone, androstenedione, adrenosterone, dehydroepiandrosterone, oxymetholone, fluoxymesterone, methandrostenolone, testolactone, pregnenolone, 17α-methylnortestosterone, norethandrolone, dihydrotestosterone, danazol, androsterone, nandrolone, stanozolol, ethylestrenol, oxandrolone, bolasterone, mesterolone, testosterone propionate, testosterone cypionate, testosterone phenylacetate, and testosterone enanthate, testosterone acetate, testosterone buciclate, testosterone heptanoate, testosterone decanoate, testosterone caprate, testosterone isocaprate, as well as esters, derivatives, prodrugs, and isomers thereof.

Although hormonal treatments are very effective at alleviating VMS, they are not appropriate for all patients. In particular, hormone therapy is usually not recommended for patients with or at risk for hormonally sensitive cancers (e.g., breast or prostate cancer). Furthermore, patients with history of clotting or severe migraines are averse to undergoing hormonal therapy because other estrogen-mediated side effects (e.g., uterine cancer, vaginal bleeding, and vein thrombosis) may emerge. Accordingly, in certain embodiments of the present invention, ODV topical compositions to be used for the treatment of vasomotor symptoms further comprise one or more non-hormonal pharmacologically active agents. Examples of suitable non-hormonal agents include, but are not limited to, steroids, α-adrenergic agonists, and β-blockers. Specific examples include bellargal (i.e., a combination of phenobarbital, ergotamine, and belladonna; T. B. Lebherz, Obstet. Gynecol., 1969, 33: 795-799), clonidine (R. M. Goldberg et al., J. Clin. One., 1994, 12: 155-158; C. L. Loprinzin et al., J. Urol., 1994, 151: 634-636), mirtazapine (M. D. Waldinger et al., Maturitas, 2000, 36: 165-168), trazadone (F. Pansini et al., Clin. Exp. Obstet. Gynecol., 1995, 22: 341-344), gabapentin (T. J. Guttuso, Neurology, 2000, 54: 2161-2163), veralipride (A. David, Am. J. Obstet. Gynecol., 1988, 158:1107-1115: P. Vercellini et al., Gynecol. Obstet. Invest., 1992, 34: 102-104), methyldopa (M. G. Hammond, J. Clin. Endocrinol. Metab., 1984, 58: 1158-1160; O. Andersen, Acta Obstet. Gynecol. Scand., 1986, 65: 405-409; B. I. Nesheim, Eur. J. Clin. Pharmacol., 1981, 20: 413-416.), bromocryptine (B. Scoccia et al., J. Clin. Endocrinol. Metab, 1988, 66: 868-871), and domperidone (L. Zichella et al., Maturitas, 1986, 8: 229-237).

Other pharmacologically active compounds and substances suitable for incorporation into ODV topical compositions of the present invention can be found in the “Physicians' Desk Reference”, 55^{th} Ed., 2001 Medical Economics Co., Inc.: Montvale, N.J., which is incorporated herein by reference in its entirety. For most or all of these agents, recommended effective dosages and regimes are known in the art.

IV—Uses of ODV Topical Compositions

According to the present invention, ODV topical compositions are useful for treating a variety of diseases, disorders or conditions. In particular, the inventive compositions can be used for the prevention, treatment or management of vasomotor symptoms (VMS) and/or pain.

In certain embodiments, an inventive ODV topical composition is used for treating female patients experiencing vasomotor instability associated with either natural menopause resulting from age-related declining ovarian function or premature or artificially-induced menopause secondary to an ovariectomy, breast cancer treatment, x-ray radiation, etc. In other embodiments, an inventive ODV topical composition is used for treating male patients experiencing vasomotor symptoms associated with either age-related androgen decline or hormone deprivation resulting from treatment for prostate cancer. In still other embodiments, an inventive ODV topical composition is used to treat any male or female individual experiencing VMS not associated with menopause or androgen decline.

Alternatively or additionally, ODV topical compositions of the present invention may be used to treat any of a variety of different types of pain experienced by mammals, including humans. For example, the inventive compositions may be used to treat acute pain (short duration) or chronic pain (regularly reoccurring or persistent), whether centralized or peripheral.

Examples of acute or chronic pain that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain, or headaches such as migraines or tension headaches, or combinations of these pains. One skilled in the art will recognize that these different types of pain may overlap one another. For example, a pain caused by inflammation may also be visceral or musculoskeletal in nature.

In certain embodiments, ODV topical compositions of the present invention are used to treat or prevent pain related to or induced by any one of the following diseases, trauma or conditions: general neuropathic conditions, such as peripheral neuropathy, phantom pain, reflex-sympathetic, dystrophy, causalgia, syringomyelia, and painful scar; specific neuralgias at any location of the body, back pain, diabetic neuropathy, alcoholic neuropathy, metabolic neuropathy; inflammatory neuropathy; chemotherapy-induced neuropathy, herpetic neuralgias, traumatic ondotalgia; endodontic odontalgia; thoracic-outlet syndrome; cervical, thoracic or lumbar radiculopathies with nerve compression; cancer with nerve invasion; traumatic-avulsion injuries; mastectomy, thoracotomy pain; spinal-cord injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; connective tissue disorders, such as systemic lupus erythematosis, systemic sclerosis, polymyositis, and dermatomyositis; and inflammatory conditions, such as acute inflammation (e.g., trauma, surgery and infection) or chronic inflammation (e.g., arthritis and gout).

As will be appreciated by one skilled in the art, the compositions of the present invention may be administered alone, or, alternatively, they may be administered serially or in combination with conventional therapeutics or therapeutic regimens used in the treatment of vasomotor symptoms or pain.

V—Dosage, Administration and Packaging

A composition of the present invention may be applied to a skin or mucosal surface adjacent to a body area to be treated (e.g., area experiencing pain) for local delivery of the ODV composition and minimal absorption of the active ingredient(s) of the composition into the subject's bloodstream (e.g., to avoid or reduce systemic effect). Alternatively, topical administration of a composition of the present invention may result in absorption of at least one active ingredient of the ODV composition into the patient's bloodstream for systemic drug distribution.

Dosage

Administration of a topical ODV composition of the present invention will be in a dosage such that the amount of ODV (or pharmaceutically acceptable salt thereof) delivered is effective for its intended purpose (e.g., prevent, reduce or alleviate pain, or relieve vasomotor symptoms). As will be appreciated by one skilled in the art, the dosage will be dependent upon the nature of the condition to be treated (vasomotor symptoms or pain), the severity of the condition, the age, weight, and general health condition of the patient as well as upon the potency, bioavailability, and in vivo half-life of the components of the inventive topical composition used. These factors are readily determinable by the attending physician in the course of therapy. Alternatively or additionally, the dosage to be administered can be determined from studies using animal models for the particular type of condition being treated, and/or from animal or human data obtained from agents which are known to exhibit similar pharmacological activities. The total dose required for each treatment may be administered by multiple doses or a single dose. Adjusting the dose to achieve maximal efficacy based on these or other methods are well known in the art and are within the capabilities of trained physicians. As studies are conducted, further information will emerge regarding the appropriate dosage levels and duration of treatment of vasomotor symptoms, different types of pain, and other conditions that can benefit from administration of the inventive topical compositions.

In certain embodiments, the composition is formulated such that a unit dose contains about 5 mg to about 500 mg of ODV, or a pharmaceutically acceptable salt thereof, wherein the dose amount is calculated based on the amount of ODV free base. For example, the unit dose may be in the range of about 25 mg to about 250 mg, or about 50 mg to about 200 mg, or about 100 mg ODV or salt thereof, as calculated based on the amount of ODV free base.

The amount of additional pharmacologically active agents (e.g., analgesic or anti-inflammatory agents) present in a topical ODV composition of the present invention may vary depending upon the dosage recommended or permitted for the particular agent, as well as the type of condition treated and the presence and nature of other active ingredients in the composition. In general, the amount of a pharmacologically active agent present in an inventive composition or a unit dose of an inventive composition is the ordinary dosage required to obtain the desired result through local administration. Such dosages are either known to or readily determined by the skilled practitioner in the pharmaceutical and/or medical arts.

Administration

The mode of administration of a topical ODV composition of the invention will mainly depend on the form of the preparation chosen. For example, gels, lotions, creams and ointments may be manually applied or sprayed (either with a manually-activated pump or with the aid of a suitable pharmaceutically acceptable propellant) onto the surface area to be treated. Alternatively, a brush, syringe, spatula or a specifically designed container (such as tube with a narrow tip) can be used to apply an inventive composition (for example in the case of the treatment of pain resulting from a wound). Application of the composition may be performed by a medical professional or by the patient. In certain embodiments, for maximum effectiveness and increased absorption, the area to which the composition is to be administered is first cleansed, for example using an astringent, such as a standard commercial antiseptic or alcohol. The area is then allowed to dry for a few seconds, and the inventive composition is then rubbed onto the complete target area and massaged, for example, until all the composition has been absorbed.

In certain embodiments, administration of a topical ODV composition of the present invention to a skin or mucosal surface area is followed by application of a dressing or bandage to cover and protect the area (for example, in the case of a surgical wound or other types of wound) or to increase penetration of the composition. The term “dressing”, as used herein, refers to any covering designed to protect a skin area. The term includes porous and non-porous coverings, woven and non-woven coverings, absorbent coverings and occlusive coverings. In some embodiments, administration of an ODV topical composition to an open wound is followed by the use of sutures, staples, adhesive strips, or tissue adhesives to close the wound and hold the tissue together during the healing process. Alternatively, an ODV topical composition may be applied after closure of a wound.

In still other embodiments, components of the composition that are comprised in separate containers are mixed together before application to the skin surface. In yet other embodiments, components of the composition that are comprised in separate containers are applied successively to the skin or mucosa surface to be treated.

Packaging

In certain embodiments, the topical compositions of the present invention are packaged as kits. A kit according to the present invention may comprise a container (e.g., a jar, tube, or other type of recipient) comprising the composition and instructions for using the composition for the treatment of vasomotor symptoms and/or pain. Alternatively, the kit may comprise a container containing a composition of the present invention, and at least one dressing, wherein the dressing is to be applied to cover the area following local administration of the composition. In certain embodiments, pharmacologically active agents may be incorporated into or coated onto the dressing. Incorporation of pharmacologically active agents into a dressing or coating of a dressing with pharmacologically active agents may be performed by any suitable method (for example, by dipping the dressing in or spraying the dressing with a solution or dispersion of the agent(s) or by applying the agent(s) in the form of a powder to the dressing). Alternatively, a kit according to the present invention may comprise two separate containers, with the first container comprising the inventive composition or some components of the inventive composition admixed with one or more physiologically acceptable carriers or excipients, and the second container comprising other components of the composition and/or a suitable medium intended to be added to the first container before use in order to obtain a ready-to-use composition.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A topical composition comprising a therapeutically effective amount of ODV, or a pharmaceutically acceptable salt thereof, and at least one physiologically acceptable carrier or excipient.

2. The topical composition of claim 1, wherein the composition is a formulation selected from the group consisting of ointment, cream, lotion, paste, gel, spray, aerosol, and oil.

3. The topical composition of claim 1, wherein the composition is a cream or a gel.

4. The topical composition of claim 1, wherein the at least one physiologically acceptable carrier or excipient is selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

5. The topical composition of claim 1 further comprising at least one absorption enhancer.

6. The topical composition of claim 5, wherein the at least one absorption enhancer is selected from the group consisting of pentadecalactone, 1,3-dioxalanes, 1,3-dioxanes, and any combination thereof.

7. The topical composition of claim 1 further comprising a therapeutically effective amount of at least one pharmacologically active agent.

8. The topical composition of claim 7, wherein the at least one pharmacologically active agent is selected from the group consisting of analgesics, anesthetics, muscle relaxants, neurotransmitter regulating agents, nociceptic agents, pre-menstrual medications, anti-depressants, anti-bipolar agents, anti-schizophrenic agents, tranquilizers, soporific agents, anti-migraine agents, skin temperature lowering products, anti-cancer agents, alkaloids, anti-metastatic agents, blood pressure controlling agents, hormones, steroids, anti-inflammatory agents, anti-ischemic agents, anti-arrhythmic agents, vitamins, minerals, anti-angiogenic agents, wound healing agents, cytokines, growth factors, anti-histaminic agents, anti-bacterial agents, anti-viral agents, antibiotics, counteracting appetite suppressants, dermatological agents such as skin renewal agents, sun screen and emollients, libido altering agents, laxatives, anti-diarrheic agents, antipruritic agents, antipyretic agents, immunostimulating agents, agents suitable for the treatment of prophylaxis diseases and conditions associated or accompanied with pain and inflammation, and any combination thereof.

9. The topical composition of claim 1, 5 or 7, wherein the therapeutically effective amount of ODV, or a pharmaceutically acceptable salt thereof, is between about 5 mg and about 500 mg, between about 25 mg and about 250 mg, or between about 50 mg and about 200 mg, wherein the amount is calculated based on the amount of ODV free base.

10. The topical composition claim 1, 5 or 7, wherein the therapeutically effective amount of ODV, or a pharmaceutically acceptable salt thereof, is about 100 mg, wherein the amount is calculated based on the amount of ODV free base.

11. A method of treating vasomotor symptoms in a subject, the method comprising administering to the subject a therapeutically effective amount of the composition of claim 1, 5 or 7.

12. The method of claim 11, wherein the subject suffering from vasomotor symptoms experiences hot flashes.

13. The method of claim 12, wherein administering the composition to the subject comprises applying a therapeutically effective amount of the composition to one or more skin surface areas of the subject's body experiencing hot flashes.

14. The method of claim 12, wherein the subject is human.

15. The method of claim 14, wherein the subject is a female patient, and the vasomotor symptoms are associated with natural menopause, chemically-induced menopause, or surgically-induced menopause.

16. The method of claim 14, wherein the subject is a female patient who is receiving or has received breast cancer treatment.

17. The method of claim 16, wherein the breast cancer treatment comprises administration of tamoxifen.

18. The method of claim 14, wherein the subject is a male patient who is naturally, chemically or surgically andropausal.

19. The method of claim 18, wherein the male patient is or has been treated for prostate cancer.

20. A method of treating pain in a subject, the method comprising administering to the subject a therapeutically effective amount of the composition of claim 1, 5 or 7.

21. The method of claim 20, wherein administering the composition to the subject comprises applying a therapeutically effective amount of the composition to one or more areas of the subject's body experiencing pain.

22. The method of claim 21, wherein the pain experienced by the subject is nociceptive pain.

23. The method of claim 21, wherein the pain experienced by the subject is neuropathic pain.

24. The method of claim 21, wherein the subject is human.
